(19) United States
(12) Patent Application Publication
(10) Pub. No.: US 2025/0302185 A1
(43) Pub. Date: Oct. 2, 2025

(54) LIGHT-EMITTING HYGIENE DEVICE USING PCB-ATTACHED MOTOR

(71) Applicant: Hee Chan CHA, Seoul (KR)

(72) Inventor: Hee Chan CHA, Seoul (KR)

(21) Appl. No.: 18/864,856

(22) PCT Filed: May 9, 2023
(86) PCT No.: PCT/KR2023/006240

§ 371 Date: Nov. 11, 2024

(30) Foreign Application Priority Data
May 9, 2022 (KR) 10-2022-0056892

(51) Int. Cl. A46B 15/00; A61L 2/00

(52) U.S. Cl. CPC A46B 15/0016; A61L 2/0052; A46B 2200/1066

(57) ABSTRACT

The present invention relates to a light-based sanitation device using a PCB-mounted motor. The device consists of a main body, a PCB arranged inside the main body, a power button in the handle area, and a light source unit in the head area. A vibration motor is attached to the upper end of the handle section and soldered to the PCB via soldering. A toothbrush head is fastened to the light source unit, with at least one toothbrush bristle formed using a waveguide. This configuration reduces noise, improves vibration stability, and enhances user convenience. The device also simplifies the manufacturing process by configuring the existing PCB with multiple or segmented structures into a single-piece structure, leading to cost reduction and potential improvements in productivity.

### TECHNICAL FIELD

The present invention relates to a light irradiation hygiene device using a printed circuit board (PCB)-attached motor. More specifically, it pertains to a design for an electric toothbrush that incorporates a vibration motor attached directly to a PCB. This structure reduces noise and improves vibration stability, contributing to the enhancement of hygiene functionality in the device.

### BACKGROUND ART OF THE INVENTION

Generally, to maintain dental hygiene, methods such as brushing with a toothbrush to remove oral bacteria-causes of plaque and tartar formation-are commonly used. Additionally, chemical antibacterial agents, such as mouthwash solutions, are often applied after brushing.

Many electric toothbrushes have been developed to enable more effective brushing. Moreover, as conventional brushing alone may not adequately eliminate bacteria, electric toothbrushes that emit light at specific wavelengths to enhance bacterial removal are also being introduced.

However, most commonly used methods rely solely on the chemical removal of bacteria through mouthwashes. While electric toothbrushes emitting white, blue, or red LED light have recently been introduced in large numbers, there remains a limitation: these devices do not specify effective methods for suppressing oral bacteria in detail.

Specifically, conventional electric toothbrushes are equipped with a vibration motor and antimicrobial light irradiation functionality. In these devices, the vibration motor is typically positioned in a plastic housing, physically separated from the circuit board, and assembled. The configuration usually involves three separate printed circuit boards (PCBs): one for the light-emitting source, one for the connection to the vibration motor, and one for the battery. As a result, the conventional design requires a three-piece PCB structure, leading to increased costs and manufacturing complexity. Additionally, these devices suffer from increased noise and reduced vibration stability during use. The prior art is disclosed in Korean Registered Patent Publication No. 10-0773210.

### CONTENT OF THE INVENTION

### Problem to Be Solved

The present invention is proposed to address the problems mentioned above in the conventional methods. The invention includes: a main body forming the housing exterior of the handle and head portions; a PCB that is shaped to match the form of the main body and positioned within it, wherein a power button is formed in the handle portion and a light-emitting unit including multiple light sources is formed in the head portion, which irradiates light through a waveguide; a vibration motor mounted on the handle portion of the main body, attached to the PCB through soldering; and a brush head plate that includes bristles and is positioned above the light-emitting unit, secured to the PCB, and formed with at least one waveguide. By utilizing the PCB-attached vibration motor, the invention aims to reduce noise, enhance vibration stability, and improve ease of use through noise reduction and increased vibration stability, thereby providing a light irradiation hygiene device using a PCB-attached motor.

Furthermore, the present invention simplifies the manufacturing process and reduces costs by configuring the conventional multiple or split PCB structure into a single-piece design. This structure also enables improved productivity, thus providing another objective of offering a light irradiation hygiene device using a PCB-attached motor.

### Means for Solving the Problem

The light irradiation hygiene device using a PCB-attached motor, according to the features of the present invention, is designed to achieve the above-mentioned objectives as follows:

A light irradiation hygiene device using a PCB-attached motor, comprising:
A main body forming the housing exterior of the handle and head portions;
A PCB configured to match the shape of the main body and positioned inside it, wherein a power button is formed in the handle portion, and a light-emitting unit comprising multiple light sources is formed in the head portion, which irradiates light through a waveguide.
A vibration motor, which is positioned in the upper area of the handle portion of the main body and attached to the PCB through soldering; and
Positioned above the light-emitting unit formed in the head portion of the PCB and secured thereto, the device includes a brush head plate equipped with bristles formed from at least one waveguide, as a structural feature.

Preferably, the PCB is configured as a single-piece structure that can be positioned inside, corresponding to the shape of the main body.

Preferably, the PCB is further configured to include a battery connector formed within the handle portion of the main body.

Preferably, the light-emitting unit of the PCB includes a first light source that irradiates short-wavelength light and a second light source that irradiates long-wavelength light. The plurality of light sources can be configured as a set that includes at least one first light source emitting short-wavelength light and a set that includes at least one second light source emitting long-wavelength light.

More preferably, the light-emitting unit is configured as a structure in which multiple light sources are arranged in a repeated pattern.

More preferably, the light-emitting unit includes a first light source that irradiates short-wavelength light and a second light source that irradiates long-wavelength light, wherein the short-wavelength light emitted by the first light source is blue light, and the long-wavelength light emitted by the second light source is red light.

More preferably, the light-emitting unit can be composed of the first light source and the second light source as LED (Light Emitting Diode) or laser diodes.

More preferably, the brush head plate includes bristles formed from at least one waveguide, wherein the bristles formed from the waveguide can be made of optical fibers.

### Effects of the Invention

According to the light irradiation hygiene device using a PCB-attached motor proposed in the present invention, the device is structured with a main body forming the housing exterior of the handle and head portions. The PCB is configured to match the shape of the main body and is positioned inside, with a power button formed in the handle portion and a light-emitting unit comprising multiple light sources that irradiate light through a waveguide formed in the head portion. The vibration motor is positioned in the upper area of the handle portion of the main body and is attached to the PCB through soldering. The brush head plate, secured to the upper part of the light-emitting unit in the head portion of the PCB, includes bristles formed from at least one waveguide. This configuration reduces noise through the use of the PCB-attached vibration motor and enhances the convenience of use by reducing noise and improving vibration stability.

Furthermore, according to the light irradiation hygiene device using the PCB-attached motor of the present invention, by configuring the conventional multiple or split PCB structure into a single-piece design, the manufacturing process can be simplified, leading to cost reduction and improved productivity.

### DESCRIPTIONS OF THE DRAWINGS

FIG. 1 is a diagram showing the configuration of the light irradiation hygiene device using a PCB-attached motor according to an embodiment of the present invention, represented as a functional block.

FIG. 2 is a diagram showing the configuration of the main body of the light irradiation hygiene device using a PCB-attached motor according to an embodiment of the present invention, represented as a functional block.

FIG. 3 is a diagram showing the PCB configuration of the light irradiation hygiene device using a PCB-attached motor according to an embodiment of the present invention, represented as a functional block.

FIG. 4 is a perspective view showing the overall configuration of the light irradiation hygiene device using a PCB-attached motor according to an embodiment of the present invention.

FIG. 5 is a diagram showing the relationship between the light-emitting unit of the PCB and the position of the brush head plate in the light irradiation hygiene device using a PCB-attached motor according to an embodiment of the present invention.

FIG. 6 is a diagram showing the configuration of the light irradiation hygiene device using a PCB-attached motor according to an embodiment of the present invention, with the light-emitting unit and the brush head plate assembled.

FIG. 7 is a plan view showing the configuration of the brush head plate of the light irradiation hygiene device using a PCB-attached motor according to an embodiment of the present invention.

### DETAILED DESCRIPTION OF THE INVENTION

Hereinafter, with reference to the attached drawings, a preferred embodiment of the present invention will be described in detail to enable those skilled in the art to easily carry out the invention. However, when explaining the preferred embodiment in detail, if a specific description of related known functions or configurations is determined to unnecessarily obscure the essence of the invention, such detailed explanations will be omitted. Furthermore, identical reference numerals will be used throughout the drawings for parts with similar functions and operations.

Furthermore, in the entire specification, when it is stated that a part is “connected” to another part, this includes not only the case of being “directly connected,” but also the case where the parts are “indirectly connected” through another component. Additionally, when a component is said to “include” another, it means that the component may include additional elements unless there is an explicit statement to the contrary.

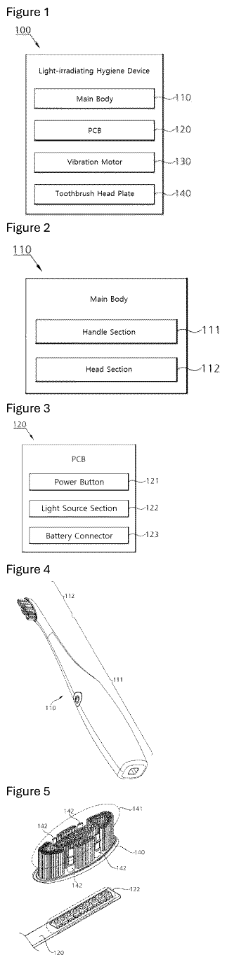

FIG. 1 is a diagram showing the configuration of the light irradiation hygiene device using a PCB-attached motor according to an embodiment of the present invention, represented as a functional block. FIG. 2 is a diagram showing the configuration of the main body of the light irradiation hygiene device using a PCB-attached motor according to an embodiment of the present invention, represented as a functional block. FIG. 3 is a diagram showing the PCB configuration of the light irradiation hygiene device using a PCB-attached motor according to an embodiment of the present invention, represented as a functional block. FIG. 4 is a perspective view showing the overall configuration of the light irradiation hygiene device using a PCB-attached motor according to an embodiment of the present invention. As shown in FIGS. 1 to 4, the light irradiation hygiene device (100) using a PCB-attached motor according to an embodiment of the present invention can be configured to include the main body (110), PCB (120), vibration motor (130), and toothbrush head plate (140).

The main body (110) is the structure that forms the housing exterior of the handle portion (111) and the head portion (112). The handle portion (111) of the main body (110) is the part where the user holds the toothbrush, and the power button (121) may be exposed. The head portion (112) of the main body (110) is equipped with the brush bristles (141) and massage bristles (142) formed from waveguides (to be described later), as well as the light-emitting unit (122). The light emitted from the light source can be delivered to the user's teeth or gums through the bristles (141).

The PCB (120) is configured to match the shape of the main body (110) and is arranged inside the main body (110). It includes a power button (121) formed in the handle portion area and a light source unit (122) containing multiple light sources, which emit light through a waveguide in the head portion area. This PCB (120) can be structured as a one-piece structure that fits inside the main body (110), corresponding to its shape. Unlike conventional PCBs, which are made of multiple or segmented structures, the one-piece structure of the PCB (120) simplifies the manufacturing process and reduces costs.

Additionally, the PCB (120) may further include a battery connector (123) formed inside the handle portion (111) of the main body (110). The light source unit (122) of the PCB (120) includes a first light source (122a) that emits short-wavelength light and a second light source (122b) that emits long-wavelength light. The multiple light sources can be configured as a set that includes at least one first light source (122a) emitting short-wavelength light and at least one second light source (122b) emitting long-wavelength light.

Furthermore, the light source unit (122) can be configured as a structure where multiple light sources are arranged repetitively. This light source unit (122) includes a first light source (122a) that emits short-wavelength light and a second light source (122b) that emits long-wavelength light. The short-wavelength light emitted from the first light source (122a) is blue light, and the long-wavelength light emitted from the second light source (122b) is red light. Additionally, the light source unit (122) can include light in the ultraviolet, infrared, or other non-visible light spectrum, such as yellow or green, in addition to red or blue light.

Additionally, the light source unit (122) may be composed of a first light source (122a) and a second light source (122b), which can be LED (Light-Emitting Diode) or laser diodes.

The vibration motor (130) is disposed in the upper region of the handle portion (111) of the main body (110) and is attached to the PCB (120) through soldering. By configuring the vibration motor (130) in an attachment structure to the PCB (120), noise caused by vibrations during the use of the electric toothbrush is reduced, and the vibration stability is improved. In other words, by attaching the motor to the PCB (120) instead of using a separate motor structure, the noise reduction and increased vibration stability enhance the ease of use. Here, the vibration motor (130) generates vibrations in the light irradiation hygiene device (100) to enable the electric toothbrush to function properly.

The toothbrush head plate (140) is positioned above the light source section (122) formed in the head portion of the PCB (120) and is fastened in place. It is configured to include at least one toothbrush bristle (141) formed as a waveguide. The toothbrush bristle (141), formed as a waveguide, can be made from optical fibers.

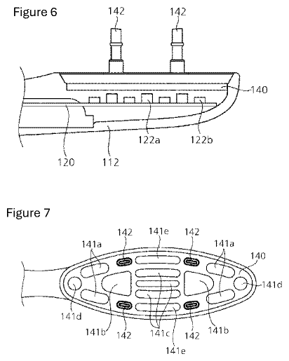

FIGS. 5 to 7 illustrate the configurations and relationships in the embodiment of the light-based sanitation device using a PCB-mounted motor according to the present invention. FIG. 5 shows the relationship between the position of the light source section on the PCB and the toothbrush head plate. FIG. 6 depicts the configuration where the light source section and the toothbrush head plate are combined. FIG. 7 displays a top-down view of the toothbrush head plate. As shown in these figures, the toothbrush bristle (141) includes at least one waveguide, which directs the light emitted from the light source to the outside.

Here, the term “waveguide” refers to a tube-like structure that has the property of transmitting light generated by the light source. The brush bristles (141) can deliver the light emitted from the light source to the user's teeth, and the brush bristles (141) using a waveguide can be made from optical fiber.

Additionally, the massage bristles (142) can also be made using a waveguide. The massage bristles (142) are used for massaging the user's gums and can transmit light emitted from the light source to massage areas such as the user's gums, inside the cheeks, or the tongue. Furthermore, to efficiently transmit the light from the light source, the massage bristles (142) may be made from a bundle of optical fibers. Since optical fibers must have a diameter below a certain threshold to transmit light effectively, the massage bristles (142) can be structured as a set of optical fiber bundles, and the capsule of the massage bristles (142) can be configured by inserting a set of optical fiber bundles.

Additionally, the toothbrush bristles (141) and massage bristles (142) formed on the toothbrush head plate (140) can be detachably mounted to the head portion (112) of the main body (110), allowing for replacement according to use.

Additionally, as shown in FIG. 7, the toothbrush bristles (141) can be configured such that the bristles (141d) located at both ends of the toothbrush head plate (140) along the longitudinal direction are longer than the other bristles (141a, 141b, 141c, 141e), in order to effectively irradiate light onto the molars.

The toothbrush bristles (141a, 141b, 141c, 141e) may also be configured in a rectangular shape according to the arrangement of the light sources. The massage bristles (142) can be positioned around the outer perimeter of an elliptical toothbrush head plate (140), placed at the virtual rectangular vertex positions, and arranged to massage the gums.

Additionally, the light source unit (122) can emit blue light to provide sterilization or teeth whitening effects in the user's oral cavity, and red light to produce effects such as anti-inflammatory action and collagen formation. Furthermore, the multiple light sources of the first light source (122a) and the second light source (122b) in the light source unit (122) can be arranged alternately, functioning to enhance the intensity of light through constructive interference.

As described above, the light-based sanitation device using a PCB-mounted motor, according to an embodiment of the present invention, includes a main body that forms the housing of the handle and head, and a PCB configured to correspond to the shape of the main body and placed inside it. The handle area of the main body includes a power button, and the head area includes a light source unit with multiple light sources that emit light through a waveguide. Additionally, the PCB, which is positioned in the head section of the main body, contains a vibrating motor attached via soldering, and the light source unit is located above the PCB's head area and secured in place. The device also includes a brush head that incorporates at least one waveguide, through which the light is transmitted.

By using a PCB-mounted vibrating motor, the device can reduce noise and improve vibration stability, resulting in decreased noise and enhanced user convenience. Furthermore, by employing a one-piece structure for the PCB, as opposed to multiple or split structures, the manufacturing process is simplified, leading to cost reduction and increased production efficiency.

The invention described herein can be modified or applied in various ways by those skilled in the art within the scope of the technical concept. The scope of the invention should be defined by the claims set forth below.

### Description of Reference Numerals

100: Light-based sanitation device according to an embodiment of the present invention
110: Main body
111: Handle section
112: Head section
120: PCB
121: Power button
122: Light source section
122a: First light source
122b: Second light source
123: Battery connector
130: Vibration motor
140: Toothbrush head Plate
141: Toothbrush bristles
141a, 141b, 141c, 141d, 141e: Toothbrush bristles
142: Massage bristles

1. A light-irradiating hygiene device (100) using a PCB (Printed Circuit Board)-mounted motor includes a main body unit (110) that forms the external housing of the handle part (111) and the head part (112). The PCB (120) is shaped to correspond to the main body unit (110) and is placed inside it. A power button (121) is positioned in the handle area, and a light source unit (122), which emits light through a waveguide and includes multiple light sources, is positioned in the head area. The vibration motor (130) is located in the upper area of the handle part (111) of the main body unit (110) and is attached to the PCB (120) through soldering. The toothbrush head plate (140) is secured above the light source unit (122) in the head area of the PCB (120) and includes toothbrush bristles (141) formed with at least one waveguide.

2. The light-based sanitation device using a PCB-mounted motor according to claim 1, wherein the PCB (120) is configured as a one-piece structure that can be disposed inside the main body (110) to correspond to the shape of the main body (110).

3. The light-based sanitation device using a PCB-mounted motor according to claim 1, wherein the PCB (120) further includes a battery connector (123) formed and disposed inside the handle portion (111) of the main body (110).

4. The light-based sanitation device using a PCB-mounted motor according to any one of claims 1 to 3, wherein the light source part (122) of the PCB (120) includes a first light source (122a) for emitting short-wavelength light and a second light source (122b) for emitting long-wavelength light, and the plurality of light sources is configured as a set comprising at least one first light source (122a) for emitting short-wavelength light and at least one second light source (122b) for emitting long-wavelength light.

5. The light-based sanitation device using a PCB-mounted motor according to claim 4, wherein the light source part (122) is configured as a structure in which a plurality of light sources are arranged in a repeating pattern.

6. The light-based sanitation device using a PCB-mounted motor according to claim 4, wherein the light source part (122) includes a first light source (122a) that emits short-wavelength light and a second light source (122b) that emits long-wavelength light, and the short-wavelength light emitted by the first light source (122a) is blue light, and the long-wavelength light emitted by the second light source (122b) is red light.

7. The light-based sanitation device using a PCB-mounted motor according to claim 4, wherein the first light source (122a) and the second light source (122b) are composed of an LED (Light-Emitting Diode) or a laser diode.

8. The light-based sanitation device using a PCB-mounted motor according to claim 4, wherein the toothbrush head plate (140) includes at least one toothbrush bristle (141) formed with a waveguide, and the toothbrush bristle (141) formed with the waveguide is made of optical fiber.
